(19) United States
(12) Patent Application Publication
(10) Pub. No.: US 2019/0223746 A1
(43) Pub. Date: Jul. 25, 2019

(54) Systems and Methods for Analyzing Brain Activity and Applications Pertaining to Augmented Reality

(71) Applicant: NeuroSteer Ltd., Ramat Gan (IL)

(72) Inventor: Nathan Intrator, Ramat Gan (IL)

(21) Appl. No.: 16/257,906

(22) Filed: Jan. 25, 2019

Related U.S. Application Data
(60) Provisional application No. 62/621,759, filed on Jan. 25, 2018.

(51) Int. Cl. A61B 5/0476; G06T 19/00; A61B 5/053; G06F 3/01

(52) U.S. Cl. CPC A61B 5/0476; G06F 3/011; A61B 5/0533; G06T 19/006

(57) ABSTRACT

An augmented reality system and methods for improving augmented reality are presented herein. Such systems are configured to monitor the brain state of an individual and configured to create a feedback loop whereby the brain state of the individual modulates parameters of the augmented reality system, thereby changing the augmented reality presented to the individual and tailoring the augmented reality presented to the individual in real time to suit the particular needs of the individual at any particular moment in time. Also presented herein are methods designed to monitor the brain state of an individual and create a feedback loop whereby the brain state of the individual modulates parameters of the augmented reality system, thereby changing the augmented reality presented to the individual and tailoring the augmented reality presented to the individual in real time to suit the particular needs of the individual at any particular moment in time.

### RELATED APPLICATIONS

This application claims priority of U.S. Provisional Application No. 62/621,759, filed Jan. 25, 2018, the entirety of which is incorporated herein by reference for all purposes.

### FIELD OF THE INVENTION

The present invention relates to a system and method for monitoring the brain state of an individual and creating a feedback loop whereby the brain state of the individual modulates responses of the system (e.g., a content suggestion system) implemented to provide augmented reality for the individual. In a particular embodiment, the present invention relates to an augmented reality system that responds to an individual's brain activity in real time and is capable of altering at least one parameter of the augmented reality provided to the individual in response to the individual's brain activity.

### BACKGROUND OF THE INVENTION

Electroencephalography (EEG) is one method to monitor electrical activity of the brain. It is typically noninvasive, with the electrodes placed along the scalp, however, invasive electrodes may be used in specific applications. EEG measures voltage fluctuations resulting from ionic current within the neurons of the brain. However, the sensitivity of the EEG electrodes limits detection to small regions of the brain, close to each electrode, thus limiting the spatial resolution of EEG. Readouts from larger brain volumes can be obtained by increasing the sensitivity of detection. Recent technological advances in electronic chip technology have been shown to increase the sensitivity of sensing electronics, such as EEG electrodes.

### SUMMARY OF THE INVENTION

Optimized mixed reality as described herein relates to a mixed reality technology that offers a new way to interact with someone's reality in real time and in a way that enhances the reality experience. Enhancing the reality experience is a new challenge that differs from and builds upon, for example, optical technological innovations and advances based solely on information access.

The present invention relates at least in part to an approach to ranking and prioritizing content or information items. Ranking, as described herein, is adapted to the mixed reality setting and to other relevant settings. In a virtual reality scenario, a content provider controls the content together with the user and is aware of the contents that the user chooses to access. In a particular embodiment, a user is currently reading a web page, which was provided by the content provider at the request of the user. Given that information, the content provider can predict what the user may choose to investigate next, and thus, can add unsolicited contents (e.g., advertisements) based on the user profile and the content in front of the user. However, in a mixed reality scenario, the content provider cannot fully know what the user is experiencing (e.g., seeing, hearing, smelling, tasting, and/or feeling, engagement, cognitive state, and/or fatigue). In such a scenario, the ability of the content provider is impaired by limited information, which renders providing an optimal unsolicited content more challenging. To bridge the information gap under such a scenario, additional information is required.

The present invention provides a system and interface whereby additional information is acquired by the content provider and thus, can help optimize unsolicited contents offered by the content provider in a mixed reality scenario, thereby providing an optimized mixed reality. Optimized mixed reality as described herein is based on the ability to scan brain activity and interpret the activity automatically in real time and have that information conveyed to the content provider, which in turn may modify its indicators/directives to suit the user's desires/needs as reflected by the user's brain activity.

In a particular embodiment, the interpretation of the user's brain activity may include detecting levels of:
Mood and anxiety
Attention/engagement and fatigue
User's excitement from previously provided contents or from identified objects in the visual environment
Excitement resulting from audio visual (real or augmented) stimulation.

In an embodiment, an augmented reality system is presented, comprising:

(i) at least one augmented reality device, wherein the at least one augmented reality device is configured to create a plurality of augmented reality environments;
(ii) at least one device configured to measure brain activity of a subject and collect particular electrical signal data representative of the brain activity of the subject;
(iii) a processor configured, when executing a set of software instructions stored in a non-transient computer-readable hardware storage medium, to interpret brain activity of the subject, wherein the processor configured to interpret brain activity performs at least the following operations:
1) projecting, in real time, the collected particular electrical signal data representative of the brain activity of the subject onto a pre-determined ordering of a denoised optimal set of wavelet packet atoms to obtain a particular set of projections of the subject;
2) normalizing, in real time, the particular set of projections of the subject using the pre-determined set of normalization factors to form a particular set of normalized projections of the subject;
3) applying at least one machine learning algorithm to the particular set of normalized projections of the subject to determine, in real time, at least one particular normalized projection in the particular set of normalized projections which corresponds to the subject's electrical brain activity, wherein the processor is configured to determine the subject's electrical brain activity from the particular set of normalized projections of the subject;
4) generating, in real time, an indication of the electrical brain activity of the subject, wherein the indication correlates with an interpretation of the electrical brain activity of the subject; and
5) causing, based on the interpretation of the brain activity, a change in at least one parameter of the at least one augmented reality device, whereby the at least one augmented reality device is configured to alter, based on the change, a first augmented reality environment to present a second augmented reality environment to the subject, thereby changing, in real time, the augmented reality environment in response to the subject's brain activity.

In a particular embodiment, the augmented reality system further comprises at least one sensor device configured to measure a physiological response distinct from brain activity. In a more particular embodiment thereof, the augmented reality system further comprises a stimulating device, wherein the stimulating device is configured to stimulate the subject's brain to alter the subject's brain activity or alter a feature presented in an augmented reality environment. More particularly, the feature presented by the augmented reality environment comprises a text, a photograph, an auditory stimulation, a video, a tactile, smell or a combination thereof. Still more particularly, the text, audio, photographs, or videos or any combination thereof comprise/s an advertisement, a reminder, or directions to particular real world destination.

In another particular embodiment of the augmented reality system, the physiological response distinct from brain activity comprises at least one of heart rate, heart rate variability, temperature, blood oxygen level, galvanic skin response (GSR), electro dermal activity, perspiration, olfactory response, blood sugar levels, hormonal level of oxytocin and cortisol, or any combination thereof.

In another particular embodiment of the augmented reality system, causing the change in the at least one parameter of the at least one augmented reality device is further based on measurements of the physiological responses distinct from brain activity and indicative of the physiological state of the subject.

In another particular embodiment of the augmented reality system, the processor configured to interpret brain activity detects an emotional state of the subject.

In another particular embodiment of the augmented reality system, specific parameters such as stress which can be measured by interpretation of brain activity in accordance with the disclosure of U.S. Pat. No. 9,955,905 and/or WO 2017/212333, cortisol level in the blood, respiration rate, heart rate and heart rate variability, as well as electro dermal activity and Galvanic Skin Response (GSR) or a combination thereof is being monitored. The AR stimulation is then aimed at reducing the stress level, when it is deemed to be higher than the baseline level. The baseline level can be measured as an average of past levels for that individual or is predetermined based on the stress level of other individuals or was predetermined for that individual based on average or otherwise some quintile level from past activity. If the activity is considered to be high, by the processor, AR stimulation may attempt to reduce it. A stress reducing stimulation can include, relaxing audio-visual stimulation, or based on the environment, providing indications that can reduce stress for example, directions to the nearest information booth, to the gate of the flight, or to the nearest bathroom. Given, a specific stimulation, such as direction to an information booth, the processor continues to measures the stress level to determine whether the previous stimulation indeed reduced the stress to a predetermined level or by a certain predetermined amount. In a closed loop case, if the processor determines that the pressure level did not reach the desired value, a different AR stimulation may be provided and vice versa, until the pressure level was reduced. Otherwise, the processor may ask the individual, or a caregiver of the individual what can be done to reduce the stress.

In some embodiments, the mood of the subject (such as, for example, a developing infant) may be inferred. For example, by way of illustration, in some embodiments, mood is inferred from channels related to stress and happiness in the BAF representation. In some embodiments, channels related to stress are 1-4 negatively correlated and 34-37, 113-1 14, 119-121 positively correlated. In some embodiments, increased activity in those channels may indicate stress, anxiety or pain suffering.

In another particular embodiment of the augmented reality system, the emotional state of the subject comprises at least one of a mood. In more particular embodiments thereof, the mood comprises at least one of acceptance, joy, interest, anger, disgust, fear, sorrow, surprise, anxiety, or depression. In a still more particular embodiment, the interest correlates with at least one of an elevated degree of engagement relative to an average or baseline degree of engagement, as measured by the degree of engagement in a period preceding the stimulation, for the subject or a reduced degree of engagement relative to the average degree of engagement for the subject. Still further, the degree of engagement correlates with a response to at least one of a real world stimulus or an augmented reality stimulus.

In another particular embodiment of the augmented reality system, the augmented reality system further comprises a stimulating device, wherein the stimulating device is configured to stimulate the subject's brain to alter the subject's brain activity or alter a feature presented in an augmented reality environment. In a more particular embodiment, the feature presented by the augmented reality environment comprises a text, a photograph, a video, or a combination thereof. More particularly, the text, photographs, or videos or any combination thereof comprises an advertisement, a reminder, or directions to particular real world destination.

In another particular embodiment, the at least one augmented reality device comprises at least one of a visual augmented reality device, an auditory augmented reality device, a tactile augmented reality device, or a gravitational augmented reality device. In a more particular embodiment, the at least one of the visual augmented reality device comprises glasses, goggles, or contact lenses; the at least one of the auditory augmented reality device comprises ear buds or headphones; the at least one of the tactile augmented reality device comprises a vibrator or stimulator; and the at least one of the gravitational augmented reality device comprises a chair or simulation capsule.

In another particular embodiment of the augmented reality system, the augmented reality system further comprises an environmental sensor device, wherein the environmental sensor device is configured to detect environmental features comprising temperature, sound volume, or light intensity.

In another particular embodiment of the augmented reality system, the augmented reality system further comprises a stimulating device, wherein the stimulating device is configured to stimulate a body part of the subject other than the brain. In a more particular embodiment, the stimulating device is a vibrator. In an even more particular embodiment, the vibrator is used to achieve sexual arousal.

In another particular embodiment of the augmented reality system, the augmented reality system further comprises determining at least one effect of the change in the at least one parameter of the at least one augmented reality device by

(iv) measuring brain activity of the subject after the change in the at least one parameter of the at least one augmented reality device and collecting particular electrical signal data representative of the brain activity of the subject; and

(vi) interpreting the brain activity of the subject after the change in the at least one parameter of the at least one augmented reality device, wherein the interpreting is performed by the processor configured, when executing the set of software instructions stored in the non-transient computer-readable hardware storage medium, to interpret brain activity of the subject after the change in the at least one parameter of the at least one augmented reality device, wherein the processor configured to interpret brain activity performs at least the following operations:
1) projecting, in real time, the collected particular electrical signal data representative of the brain activity of the subject after the change in the at least one parameter of the at least one augmented reality device onto the pre-determined ordering of a denoised optimal set of wavelet packet atoms to obtain the particular set of projections of the subject after the change in the at least one parameter of the at least one augmented reality device;
2) normalizing, in real time, the particular set of projections of the subject after the change in the at least one parameter of the at least one augmented reality device using the pre-determined set of normalization factors to form a particular set of normalized projections of the subject after the change in the at least one parameter of the at least one augmented reality device;
3) applying at least one machine learning algorithm to the particular set of normalized projections of the subject after the change in the at least one parameter of the at least one augmented reality device to determine, in real time, at least one particular normalized projection in the particular set of normalized projections which corresponds to the subject's electrical brain activity after the change in the at least one parameter of the at least one augmented reality device, wherein the processor is configured to determine the subject's electrical brain activity after the change in the at least one parameter of the at least one augmented reality device from the particular set of normalized projections of the subject; and
4) generating, in real time, an indication of the electrical brain activity of the subject after the change in the at least one parameter of the at least one augmented reality device, wherein the indication correlates with an interpretation of the electrical brain activity of the subject after the change in the at least one parameter of the at least one augmented reality device; and
5) determining, based on the interpretation of the electrical brain activity of the subject after the change in the at least one parameter of the at least one augmented reality device, whether a second change in the at least one parameter of the at least one augmented reality device would improve the augmented reality environment for the subject.

In another particular embodiment of the augmented reality system, an indicator that the second change in the at least one parameter of the at least one augmented reality device resulted in presentation of an improved augmented reality environment for the subject would, for example, be a reduction in stress levels in the subject is measured by brain activity functions (BAFs).

As described herein above, in some embodiments, the mood of the subject (such as, for example, a developing infant) may be inferred. For example, by way of illustration, in some embodiments, mood is inferred from channels related to stress and happiness in the BAF representation. In some embodiments, channels related to stress are 1-4 negatively correlated and 34-37, 113-1 14, 119-121 positively correlated. In some embodiments, increased activity in those channels may indicate stress, anxiety or pain suffering.

In another particular embodiment of the augmented reality system, the content of the augmented reality environment changes based on the interpretation of the electrical brain activity of the subject after the change in the at least one parameter of the at least one augmented reality device.

In another particular embodiment of the augmented reality system, the content of the augmented reality environment which changes, based on the interpretation of the electrical brain activity of the subject after the change in the at least one parameter of the at least one augmented reality device, comprises text, photographs, or videos.

In yet another particular embodiment, a method for improving augmented reality is presented, comprising:

(i) receiving, by a processor, particular electrical signal data representative of brain activity of a subject exposed to at least one first augmented reality environment, wherein the at least one first augmented reality environment has been generated by at least one augmented reality device;
(iii) projecting, by the processor, in real time, the collected particular electrical signal data representative of the brain activity of the subject onto a pre-determined ordering of a denoised optimal set of wavelet packet atoms to obtain a particular set of projections of the subject;
(iv) normalizing, by the processor, in real time, the particular set of projections of the subject using the pre-determined set of normalization factors to form a particular set of normalized projections of the subject;
(v) applying, by the processor, at least one machine learning algorithm to the particular set of normalized projections of the subject to determine, in real time, at least one particular normalized projection in the particular set of normalized projections which corresponds to the subject's electrical brain activity, wherein the processor determines the subject's electrical brain activity from the particular set of normalized projections of the subject;
(vi) generating, by the processor, in real time, an indication of the electrical brain activity of the subject, wherein the indication correlates with an interpretation of the electrical brain activity of the subject; and
(vii) causing, by the processor, based on the interpretation of the brain activity, a change in at least one parameter of the at least one augmented reality device, whereby the at least one first augmented reality environment is changed to present at least one second augmented reality environment to the subject,
thereby improving the augmented reality environment in response to the subject's brain activity.

In a particular embodiment, the method further comprises measuring, by at least one sensor device, a physiological response distinct from brain activity. In a more particular embodiment, the physiological response distinct from brain activity comprises at least one of heart rate, heart rate variability, temperature, oxygenation, galvanic skin response (GSR), electro dermal activity, perspiration, olfactory response, blood sugar levels, or any combination thereof.

In a particular embodiment of the method, causing the change in the at least one parameter of the at least one augmented reality device is further based on measurements of the physiological responses distinct from brain activity and indicative of the physiological state of the subject.

In a particular embodiment of the method, the processor configured to interpret brain activity detects an emotional state of the subject. In a more particular embodiment, the emotional state of the subject comprises at least one of a mood. In a still more particular embodiment, the mood comprises at least one of acceptance, joy, interest, anger, disgust, fear, sorrow, surprise, anxiety, or depression. In another particular embodiment, the interest correlates with at least one of an elevated degree of engagement relative to an average degree of engagement for the subject or a reduced degree of engagement relative to the average degree of engagement for the subject. In another particular embodiment, the degree of engagement correlates with a response to at least one of a real world stimulus or an augmented reality stimulus.

In a particular embodiment of the method, the method further comprises stimulating, by a stimulating device, the subject's brain to alter the subject's brain activity or alter a feature presented in an augmented reality environment. In a more particular embodiment, the feature presented by the augmented reality environment comprises a text, a photograph, a video, or a combination thereof. In a still more particular embodiment, the text, photographs, or videos or any combination thereof comprise/s an advertisement, a reminder, or directions to particular real world destination.

In a particular embodiment of the method, the method further comprises stimulating, by a stimulating device, the subject's brain to alter the subject's brain activity or alter a feature presented in an augmented reality environment. In a more particular embodiment, the feature presented by the augmented reality environment comprises a text, a photograph, a video, or a combination thereof. In a still more particular embodiment, the text, photographs, or videos or any combination thereof comprise/s an advertisement, a reminder, or directions to particular real world destination.

In a particular embodiment of the method, the at least one augmented reality device comprises at least one of a visual augmented reality device, an auditory augmented reality device, a tactile augmented reality device, or a gravitational augmented reality device. In a more particular embodiment of the method, the at least one of the visual augmented reality device comprises glasses, goggles, or contact lenses; the at least one of the auditory augmented reality device comprises ear buds or headphones; the at least one of the tactile augmented reality device comprises a vibrator or stimulator; and the at least one of the gravitational augmented reality device comprises a chair or simulation capsule.

In a particular embodiment of the method, the method further comprises detecting environmental features, by an environmental sensor device, comprising temperature, sound volume, or light intensity.

In a particular embodiment of the method, the method further comprises stimulating, by a stimulating device, a body part of the subject other than the brain.

In another particular embodiment of the method, the method further comprises determining at least one effect of the change in the at least one parameter of the at least one augmented reality device by

(iv) measuring brain activity of the subject after the change in the at least one parameter of the at least one augmented reality device and collecting particular electrical signal data representative of the brain activity of the subject; and

(vi) interpreting the brain activity of the subject after the change in the at least one parameter of the at least one augmented reality device, wherein the interpreting, by the processor, when executing the set of software instructions stored in the non-transient computer-readable hardware storage medium, interprets brain activity of the subject after the change in the at least one parameter of the at least one augmented reality device, wherein the processor interprets brain activity by performing at least the following operations:
1) projecting, by the processor, in real time, the collected particular electrical signal data representative of the brain activity of the subject after the change in the at least one parameter of the at least one augmented reality device onto the pre-determined ordering of a denoised optimal set of wavelet packet atoms to obtain the particular set of projections of the subject after the change in the at least one parameter of the at least one augmented reality device;
2) normalizing, by the processor, in real time, the particular set of projections of the subject after the change in the at least one parameter of the at least one augmented reality device using the pre-determined set of normalization factors to form a particular set of normalized projections of the subject after the change in the at least one parameter of the at least one augmented reality device;
3) applying, by the processor, at least one machine learning algorithm to the particular set of normalized projections of the subject after the change in the at least one parameter of the at least one augmented reality device to determine, in real time, at least one particular normalized projection in the particular set of normalized projections which corresponds to the subject's electrical brain activity after the change in the at least one parameter of the at least one augmented reality device, wherein the processor determines the subject's electrical brain activity after the change in the at least one parameter of the at least one augmented reality device from the particular set of normalized projections of the subject; and
4) generating, by the processor, in real time, an indication of the electrical brain activity of the subject after the change in the at least one parameter of the at least one augmented reality device, wherein the indication correlates with an interpretation of the electrical brain activity of the subject after the change in the at least one parameter of the at least one augmented reality device; and
5) determining, by the processor, based on the interpretation of the electrical brain activity of the subject after the change in the at least one parameter of the at least one augmented reality device, whether a second change in the at least one parameter of the at least one augmented reality device caused a desired change in the at least one of the brain activity or other physiological parameters, thereby improving the augmented reality environment for the subject.

In another particular embodiment of the augmented reality system, an indicator that the second change in the at least one parameter of the at least one augmented reality device resulted in presentation of an improved augmented reality environment for the subject would, for example, be an increase in engagement or excitement and/or an improvement in mood, whereby increased engagement or excitement and/or an improved mood in the subject is measured by brain activity functions (BAFs).

In a particular embodiment of the method, the content of the augmented reality environment changes based on the interpretation of the electrical brain activity of the subject after the change in the at least one parameter of the at least one augmented reality device. In a more particular embodiment of the method, the content of the augmented reality environment which changes, based on the interpretation of the electrical brain activity of the subject after the change in the at least one parameter of the at least one augmented reality device, comprises text, photographs, or videos or any combination thereof.

Some embodiments of the invention are herein described, by way of example only. With specific reference to the examples, it is stressed that the particulars shown are by way of example and for purposes of illustrative discussion of embodiments of the invention. In this regard, the description makes apparent to those skilled in the art how embodiments of the invention may be practiced.

### BRIEF DESCRIPTION OF THE DRAWINGS

FIG. 1 shows a schematic diagram depicting an embodiment of the invention. The hatched arrow and box indicate an optional feature of systems and methods described herein.

FIG. 2 presents a list of physiological parameters/features measured in embodiments of the invention.

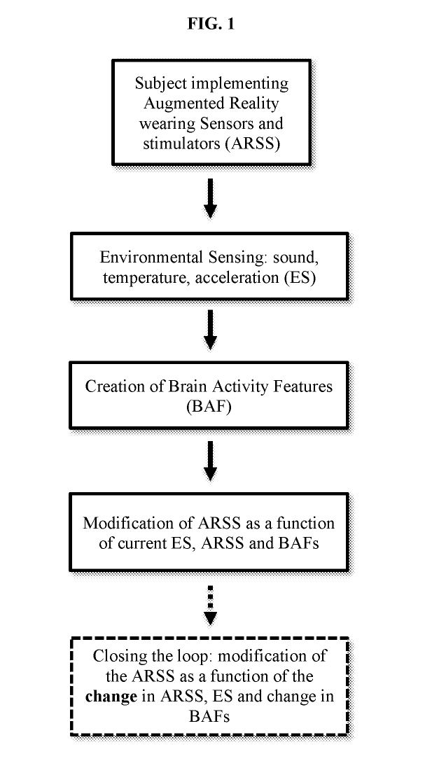

### DETAILED DESCRIPTION OF THE INVENTION

Among those benefits and improvements that have been disclosed, other objects and advantages of this invention will become apparent from the following description taken in conjunction with the disclosures of U.S. Pat. No. 9,955,905 and WO 2017/212333, the entire content of each of which is incorporated herein by reference. U.S. Pat. No. 9,955,905 and WO 2017/212333, for example, disclose how to detect brain states (e.g., stress, excitement, engagement, etc.) based on brain activity. These references disclose how to create different variants of brain activity functions (BAFs) correlating to different brain states.

The Visual Indication of at Least One Personalized Mental State of the Particular Individual: In some embodiments, the normalized, re-ordered plurality of a statistical measure of projections onto pre-determined wavelet packet atoms is assembled into a visual representation, wherein each individual normalized pre-determined wavelet packet atom in the plurality, corresponds to a BAF, and is arranged in the representation according the predetermined order. As used herein, a “BAFs representation” refers to a visual representation of the normalized, re-ordered plurality of pre-determined projections onto wavelet packet atoms.

In some embodiments, the BAFs representation of the particular individual has 121 individual BAFs. Alternatively, in some embodiments, the BAFs representation of the particular individual has up to 200 individual BAFs. Alternatively, in some embodiments, the BAFs representation of the particular individual has from 10 to 200 individual BAFs. Alternatively, in some embodiments, the BAFs representation of the particular individual has from 1 to 1000 individual BAFs. Alternatively, in some embodiments, the BAFs representation of the particular individual has from 30 to 1000 individual BAFs. Alternatively, in some embodiments, the BAFs representation of the particular individual has at least 30 individual BAFs. Alternatively, in some embodiments, the BAFs representation of the particular individual has a number of individual BAFs which is a multiple (e.g., 2×, 3×, 4×, 5×, 6×, etc.) of a number BAFs being recorded.

In some embodiments, the BAFs representation of the subject has 121 individual BAFs. Alternatively, in some embodiments, the BAFs representation of the subject over 200 individual BAFs. Alternatively, in some embodiments, the BAFs representation of the subject has from 10 to 200 individual BAFs. Alternatively, in some embodiments, the BAFs representation of the subject has from 1 to 1000 individual BAFs. Alternatively, in some embodiments, the BAFs representation of the subject has from 30 to 1000 individual BAFs. Alternatively, in some embodiments, the BAFs representation of the subject has at least 30 individual BAFs. Alternatively, in some embodiments, the BAFs representation of the subject has a number of individual BAFs which is a multiple (e.g., 2×, 3×, 4×, 5×, 6×, etc.) of a number of neural networks being analyzed. In some embodiments, the BAFs include traditional EEG recordings.

Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. In some embodiments, the terms “instantaneous,” “instantaneously,” “instantly,” and “in real time” refer to a condition where a time difference between a first time when a search request is transmitted and a second time when a response to the request is received is no more than 1 second. In some embodiments, the time difference between the request and the response is between less than 1 second and several seconds.

As used herein, the term “dynamic(ly)” means that events and/or actions can be triggered and/or occur without any human intervention. In some embodiments, events and/or actions in accordance with the present invention can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

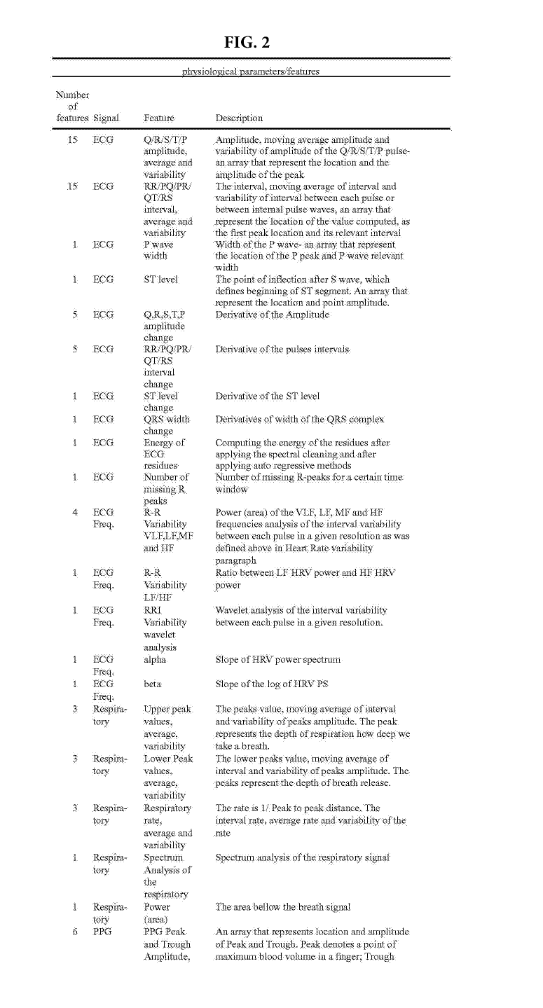

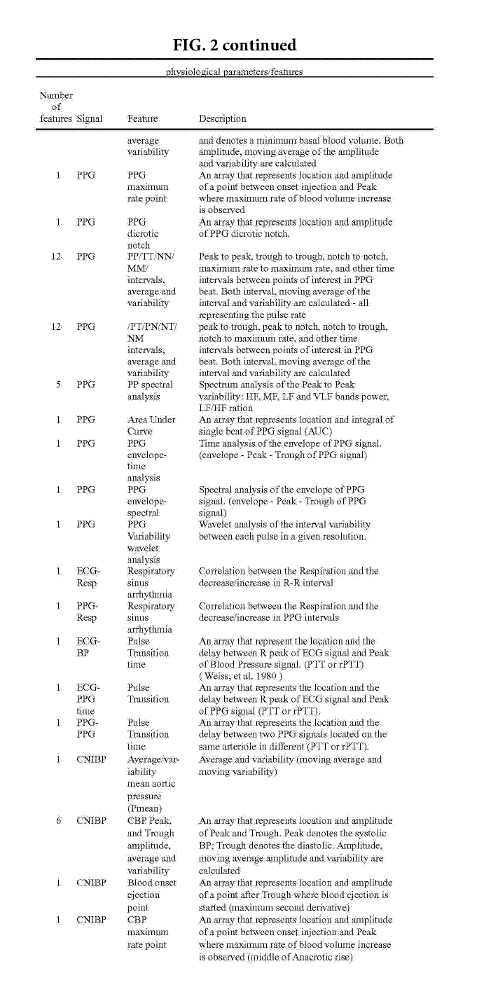

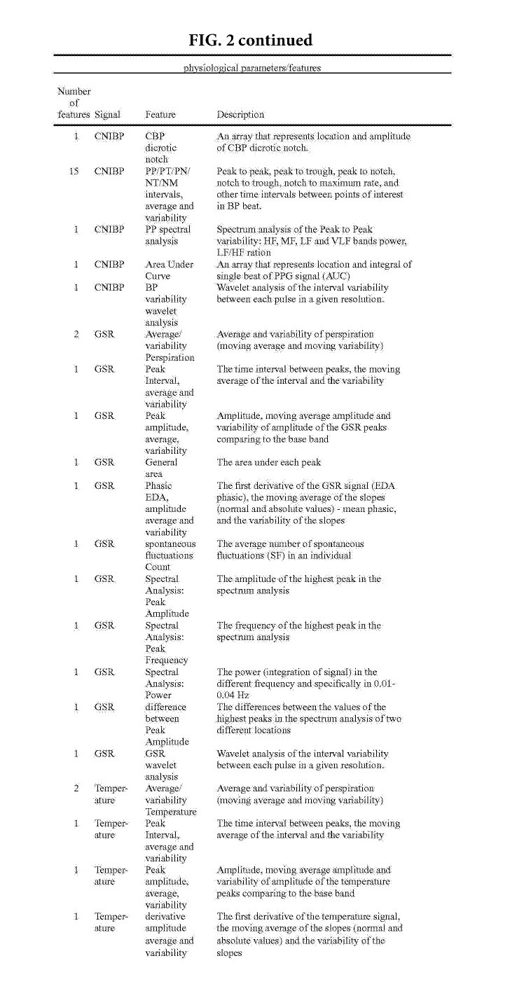

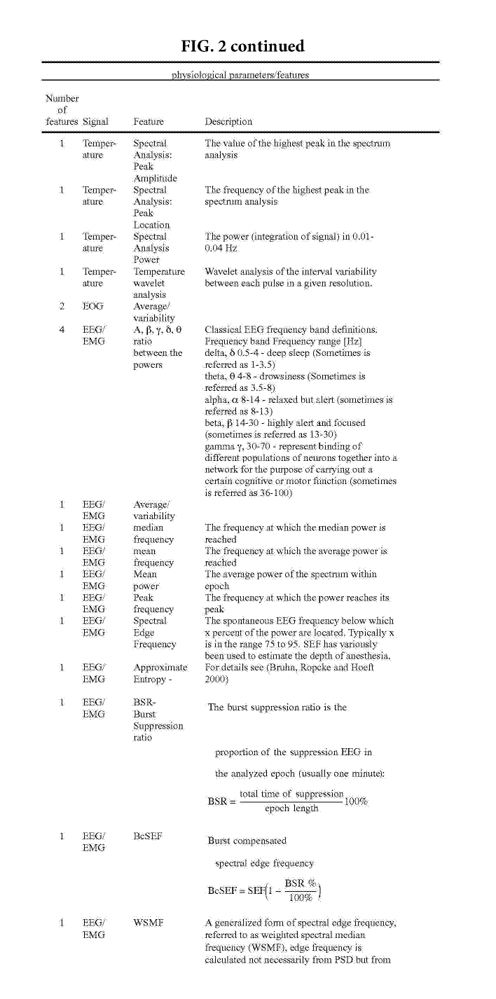

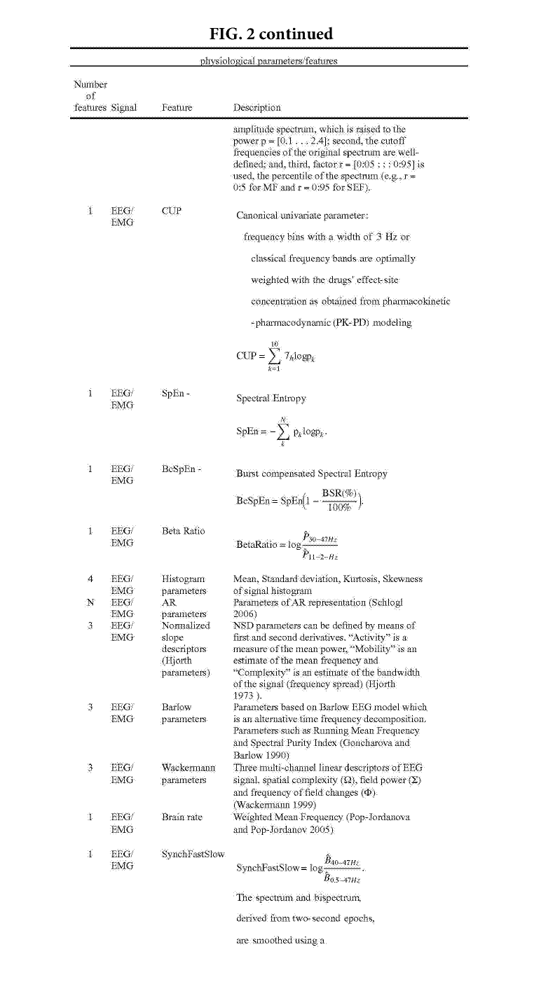

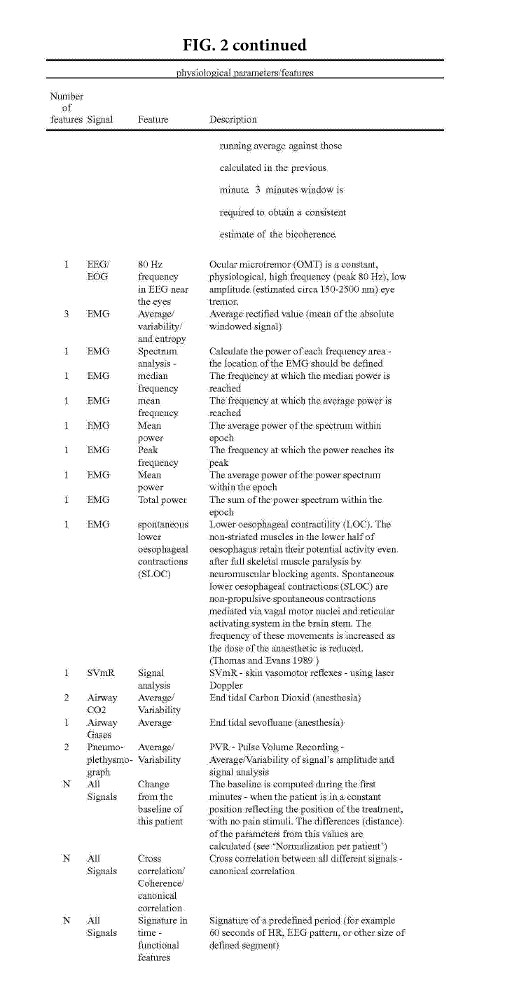

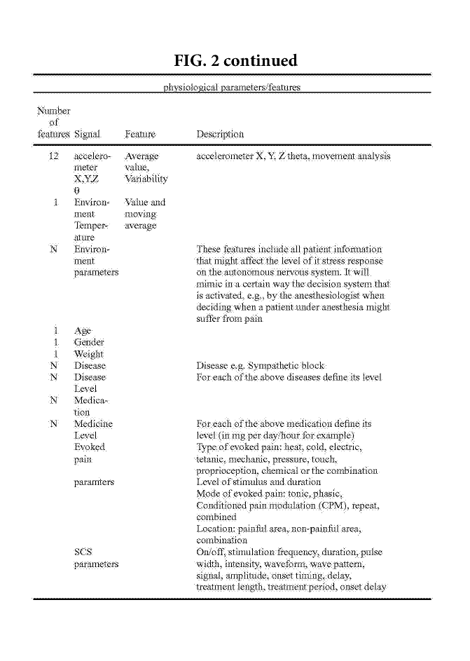

FIG. 2 presents non-limiting examples of physiological signals, including blood pressure (BP), respiration, internal and/or surface temperature, pupil diameter, galvanic skin response (GSR), and signals received and/or derived from electrocardiography (ECG), photoplethysmography (PPG), electrooculography (EOG), electroencephalography (EEG), electromyography (EMG), frontalis electromyogram (FEMG), laser Doppler velocimetry (LDV), dynamic light scattering (DLS), near-infrared spectroscopy (NIRS), partial pressure of carbon dioxide, and accelerometers or any portion or combination thereof. In a particular embodiment, a physiological signal may further comprise any signal that is measurable and/or detectable from a subject.

According to some embodiments, physiological signals may be based on parameters extracted from at least PPG and GSR signals and may for example include PPG amplitude, PPG amplitude variation, pulse rate (PR) interval, PR variability and GSR fluctuations.

As used herein, the term “Pupil Diameter Measurement (PD)” may refer to measurements of pupil size and movement. PD may be measured by infrared videography or computerized pupillometry.

As used herein, the term “Electromyography (EMG)”, refers to a technique for recording and evaluating physiologic properties of muscle activity either at rest or while contracting. EMG signals may be recorded through surface electrodes. A plurality of location specific EMG signals may be recorded from various locations on a subject and/or muscle groups. Frontalis (scalp) Electromyogram (FEMG), for example, measures the frontalis muscle underlying the forehead.

As used herein, the term “Photo PlethysmoGraph (PPG)” may refer to a non-invasive transducer configured to measure relative changes of blood volume from a finger or from other different body locations (e.g., a finger, hand, earlobe, forehead. forearm, etc.)

As used herein, the term “Electro-Cardio-Gram (ECG)” may refer to non-invasive recordings of the electrical activity of the heart.

As used herein, the term “ElectroEncephaloGram (EEG)” may refer to non-invasive readings of the electrical activity of the brain, as recorded from electrodes placed on the scalp.

As used herein, the term “ElectrogastroenteroGram (EGG)” may refer to non-invasive readings of the electrical activity of the stomach and the intestines.

As used herein, the term “Galvanic Skin Response (GSR)” may refer to non-invasive readings of the electrical conductance or resistance of the skin, which varies depending on the amount of sweat-induced moisture on the skin. Also known as Skin conductance, electro-dermal response (EDR), skin conductance response (SCR) and Galvanic skin resistance.

As used herein, the term “ElectroOculaGraph (EOG)” may refer to non-invasive recordings of electrical activity produced by eye movement and retina, as recorded from electrodes placed on the face and frontal lobe.

As used herein, the term “Blood pressure (BP)”, may refer to arterial blood pressure, i.e., to the force exerted by circulating blood on the walls of the larger arteries. BP may be measured by invasive or non-invasive methods and can be read continuously (Continuous Non Invasive Blood Pressure—CNIBP) or discretely (NIBP).

As used herein, the term “Laser Doppler Velocimetry (LDV)” may refer to quantification of blood flow in tissues such as the skin. LVD may enable calculation of parameters such as vasomotor reflex (SVMR).

As used herein, the term “Capnography” may refer to measurements of concentration or partial pressure of carbon dioxide (CO₂). Other measurements on expiratory gases may also be determined for example concentration end-tidal nitrous oxide (N₂O), oxygen (O₂), or anesthetic agents.

As used herein, the term “Accelerometer” may refer to a device for measuring movement, acceleration and gravity induced reaction forces.

In an embodiment pertaining to optimized mixed reality in a real life scenario, namely an airport. A subject (e.g., a person) is in an airport environment wearing augmented reality goggles. The at least one device configured to measure brain activity in the person and collect particular electrical signal data representative of the brain activity of the person (a brain activity sensor which may, e.g., be incorporated into the augmented reality goggles) senses that the person is stressed. Upon detection of stress in the person, the processor (or content suggestion system) chooses to present arrows indicating the locations of airport facilities that may provide comfort to the person, thereby reducing stress levels in the person. Under such a circumstance, the content suggestion system may choose to present arrows indicating the locations of the nearest bathrooms, eateries, and/or location of the flight information booth. In a more particular embodiment, the content suggestion system may choose to present arrows indicating the locations of a restaurant or bar that serves food or drinks preferred by the person under stress. The content suggestion system may further present arrows directing the person under stress to locations assigned to the particular airline on which the person is flying or, for example, the person's departure gate.

In a further embodiment, the at least one device configured to measure brain activity in the person and collect particular electrical signal data representative of the brain activity of the person (or brain activity sensor) senses that the person is excited and actively engaged in the environment of the airport. Under circumstances wherein the brain activity sensor senses excitement, engagement, or heightened interest, the content suggestion system may choose to present arrows directing the person to, for example, shopping destinations within the airport. Such shopping destinations may include duty free shops and may be accompanied by advertisements about specific items on sale. Advertisements may further be selected based on product preferences that can be determined based on, for example, the user's shopping profile. In a particular embodiment wherein the user has a history of purchasing perfumes, for example, the processor or content suggestion system could direct the user to perfumes that are on sale, especially those for which the user has a buying preference.

If there appears to be little brain excitement responsive to the indication of the arrows pointing to, for example, the duty free shops, the content suggestion system may choose to provide alternative information, including without limitation, general flight information, news, or other information based on the user's profile preferences.

In another embodiment, if there appears to be an increase in brain excitement responsive to an advertisement of a new bottle of Scotch Whiskey, an arrow indicating the location of a duty free shop that sells alcohol will be presented. The change in suggestion provided by the processor or content suggestion system is effectuated without needing active engagement on the part of the user. In other words, the content suggestion system detects the user's engagement/excitement and automatically alters the suggestions it provides to suit the user's responses.

Accordingly, optimized mixed reality as described herein does not require the user to provide feedback responsive to the content suggestion system in an active, deliberate manner by way of, for example, clicking “like” or “dislike” indicators, but rather obviates the need for such potential distractions on the part of the user by sensing the user's brain activity responsive to suggestions made by the content suggestion system.

In another embodiment an optimized mixed reality relating to changing perceived acceleration/gravitational force is presented. In this embodiment, a subject is sitting on a chair that rotates at a certain speed around an axis at or outside the center of the chair, wherein the angle of the chair with respect to the rotating axis affects the gravitational force and the perceived weight and acceleration of the subject. In this embodiment, the angle of the chair may be altered to alter the subject's perceived weight and acceleration. Such an embodiment is applicable to, for example, flight simulation exercises, such as those performed by gamers and pilots.

In another embodiment, temperature and other environmental changes may be implemented. A subject may be sitting or standing, walking or running (treadmill with speed and slope changing) inside a self-driving capsule surrounded by audio/visual and other sensory stimulation, such as those affecting temperature and humidity, smell, drinking fluids whose taste can be affected. All these parameters can be changed based on the brain and other physiological readings from the subject. For example, the type and volume of the music can be changed based on speed and slope of the treadmill and its effect on the heart rate, perspiration, and other physiological parameters to reduce heart rate for a given physical challenge via altering the musical stimulation.

In a closed loop embodiment, the parameter is modified following the change in heart rate to lead to some optimal parameter given all other environmental and physiological parameters. Such ‘optimal’ parameters are recorded for future stimulation. In one embodiment it is found that, for example, a certain temperature is optimal for activity at a certain physical challenge, and/or a certain type of music as well as visual stimulation and/or brain stimulation presents an optimized augmented reality environment conducive to peak performance.

In an embodiment directed to altering stress levels and promoting concentration, a change in background music may be used to affect stress (e.g., reduce stress) and/or enhance concentration [as is read from brain activity features (BAFs)] during a challenging cognitive task as is measured by BAFs or by an external evaluator of the subject's task, such as an evaluator of the difficulty of the content that is being read, or the cognitive challenge of a game.

In a text embodiment thereof, a person is searching the web for “inspiring poetry”. The depth and complexity of the poetry presented is correlated to the brain state of the subject searching any may be varied based on the stress levels and/or and cognitive state of the subject. For example, more complicated poetry is presented when the subject is determined to be in a more aroused cognitive activity state.

In another embodiment thereof, news items chosen from the preferred items of the subject are reordered based on the cognitive and emotional state of the subject. For example, more scientific subject matter is presented during higher brain cognitive arousal states.

In another embodiment thereof, instructions for a specific type of meditation are provided based on person's emotional and cognitive state. For example, compassion meditation is suggested during low mood (depression) states which can be determined via brain activity state, or hormone levels, such as, e.g., cortisol or oxytocin levels. Relaxing meditation can be suggested when the subject is exhibiting BAFs characteristic of high stress levels.

In another embodiment, tactile stimulation is provided as needed. In some cases of fronto-temporal dementia (FTD) or Parkinson's disease, brain activity may be higher than normal (also known in the art as increased default mode network). In such cases, it is important to provide feedback to the subject to reduce such activity since prolonged high activity may be associated with brain dysfunction and cell death, because not enough oxygen is delivered to the brain to support the elevated activity. In such cases, vibrotactile stimulation, may be used to reduce the elevated activity by itself, or may alert the subject to get into a brain state that reduces the activity (for example, via a breathing meditation, walking, or looking at animal pictures on the phone). In another embodiment, the objective is to elicit sexual arousal and pleasure in a subject. In this embodiment, sexual arousal and pleasure may be measured/assessed by brain states and hormone concentration and vibrator-induced stimulation can be modified based on reading of these parameters. Alternatively, or in addition, sexual arousal and pleasure may be enhanced further by presenting visual imagery expected to be or pre-determined to be of sexual interest to the subject.

In another embodiment, detection of abnormal electrical activity such as gate freeze in Parkinson's, hyper or hypo brain activity in cases of frontal lobe dysfunction such as Parkinson's, coma and other brain disorders, pre-ictal activity of epilepsy or migraine, epileptic seizure, migraine, and/or various types of pain may be addressed using aspects of the system and method described herein. In a particular embodiment, augmented reality may be implemented in an enhanced manner by exposing the subject afflicted with the above to soothing music to reduce the abnormal activity. The present system and methods may be used to change and optimize the soothing effect of the music by adjusting the augmented reality environment (comprising, e.g., soothing music) in accordance with BAFs of the subject.

The system and method described herein may also be used to provide enhanced treatment for various brain stimulation techniques, including without limitation, invasive techniques such as deep brain stimulation, or non invasive such as transcranial direct stimulation (tDCS), transcranial magnetic stimulation (TMS), transcranial alternating current stimulation (tACS), and others. In a particular embodiment, augmented reality may be implemented in an enhanced manner by exposing the subject undergoing one of the above stimulations with various augmented reality features (such as, e.g., exposure to soothing music, beautiful visual representations, etc.) to enhance to the therapeutic stimulation. Such enhancement may provide better efficacy and/or prolong the beneficial effects of the therapeutic stimulation. The present system and methods may be used to change and optimize the soothing/therapeutic effect of, e.g., music by adjusting the augmented reality environment (comprising, e.g., soothing music) in accordance with BAFs of the subject.

The system and method described herein may also be used to provide enhanced treatment when used in conjunction with various other medical interventions such as those involving pharmaceutical drugs, or brain and body stimulation (e.g., massage). In a particular embodiment, augmented reality may be implemented in an enhanced manner by exposing the subject undergoing one of the above medical interventions with various augmented reality features (such as, e.g., exposure to soothing music, beautiful visual representations, etc.) to enhance to the medical interventions. Such enhancement may provide better efficacy and/or prolong the beneficial effects of the medical interventions. The present system and methods may be used to change and optimize the soothing/therapeutic effect of, e.g., music by adjusting the augmented reality environment (comprising, e.g., soothing music) in accordance with BAFs of the subject.

An augmented reality system that responds to an individual's brain activity in real time and is capable of altering at least one parameter of the augmented reality provided to the individual in response to the individual's brain activity is presented herein. In an embodiment, the augmented reality system comprises:
a. Augmented reality (AR) environment; this may include: Gear VR by Samsung, HTC VIVE, Hololens by Microsoft, Oculus Rift and Go, PlayStation VR, MagicLeap.
b. Brain sensing device
c. Construction of BAFs
d. At least one parameter change in the AR, such as direction to information at the airport or to the shopping, change of advertisement on the AR screen, change of stimulation: neural, tactile and vibratory, or stimulation to other 5 basic senses, as well as temperature and acceleration.

The augmented reality system may optionally include additional physiological sensors affecting the parameter change. Such physiological sensors may detect heart rate; heart rate variability (apple watch); galvanic skin response (GSR) and electrodermal activity such as embrace 2 by empatica; temperature; sugar level; cortisol; oxygen level and saturation (pulse oxygen detector); gene expression changes; perspiration; and/or acceleration.

The augmented reality system may optionally include additional environmental sensors affecting the parameter change, including, without limitation, environmental sensors detecting background sound and noise, including the subject's breathing and other body sounds, background temperature, humidity, acceleration, oxygen level, and/or smell.

The augmented reality system may optionally include a closed loop to sense the effect of the parameter change in AR and modify the AR stimulation accordingly (e.g. if the virtual soda can put on the table did not cause excitement, try a different soda can).

The augmented reality system may optionally include AR presented as text, e.g web page, or stills/pictures, or videos, and the content changes according to brain and other physiological activity sensing and interpretation, e.g. the advertisement is changed.

The augmented reality system may optionally encompass changing the AR to affect the subject's emotional feelings such as those relating to pleasure, calmness, alertness, attention etc. mood and anxiety, engagement, excitement, fear and more.

The augmented reality system may optionally include specific stimulators such as those that achieve a tactile or other stimulation of the skin (vibro, temperature, electric).

In another embodiment, a method for detection of at least one abnormal electrical brain activity is presented, comprising:

a) utilizing a first electroencephalographic (EEG) monitoring device having a first set of three electrodes and applying the first set of three electrodes to particular points on a head of each individual of a plurality of individuals, wherein the three electrodes of the first set are:
1) a first recording electrode,
2) a second recording electrode, and
3) a reference electrode;

b) collecting, by the first EEG monitoring device, at least 100 recordings of electrical signal data representative of brain activity of the plurality of individuals to form recorded electrical data;

c) utilizing a first processor configured, when executing a first set of software instructions stored in a first non-transient computer-readable hardware storage medium, to perform at least the following operations:
1) obtaining a pre-determined ordering of a denoised optimal set of wavelet packet atoms, by:
   i) obtaining an optimal set of wavelet packet atoms from the recorded electrical signal data from the recordings from the plurality of individuals, by:
      1) selecting a mother wavelet;
      2) determining an optimal set of wavelet packet atoms, by:
         a) deconstructing the recorded electrical signal data into a plurality of wavelet packet atoms, using the selected mother wavelet;
         b) storing the plurality of wavelet packet atoms in at least one first computer data object;
         c) determining the optimal set of wavelet packet atoms using the pre-determined mother wavelet and storing the optimal set of wavelet packet atoms in at least one second computer data object, wherein the determining is via utilizing a Coifman-Wickerhauser Best Basis algorithm;
   ii) denoising the obtained optimal set of wavelet packet atoms from the recordings from the plurality of individuals;
   iii) reordering the denoised optimal set of wavelet packet atoms from the recorded electrical signal data from the plurality of individuals to obtain a pre-determined ordering of the denoised optimal set of wavelet packet atoms, by determining a minimum path based on:
      1) projecting the recorded electrical signal data onto the denoised optimal set of wavelet packet atoms to obtain a set of projections,
         wherein a projection is a result of a convolution of an electrical signal in each time window of the signal and a wavelet packet atom;
      2) determining a collection of wire lengths within the set of projections;
      3) storing the collection of wire lengths for the set of projections in at least one third computer data object;
      4) iteratively determining a plurality of (i) orders of the projections, and (ii) respective wire lengths, by:
         i) determining the wire length between every two projections by at least one of:
         1) determining either mean or sum of absolute distance of a statistical measure of an energy of each projection from adjacent projections, and
         2) 1—a correlation of every two projections onto the wavelet packet atoms; and
         ii) storing the wire length data in at least one fourth computer data object;
5) determining, from the plurality of respective wire lengths, a particular order of projections that minimizes either the mean or sum of the wire lengths across the projections, across each time window, and across all individuals within the plurality of individuals;

d) defining a set of pre-determined normalization factors, and storing the pre-determined normalization factors in at least one fifth computer data object;

e) utilizing a second EEG monitoring device having a second set of three electrodes and applying the second set of three electrodes to the particular points on a head of a particular individual;

f) collecting, by the second EEG monitoring device, in real-time, a recording of particular electrical signal data representative of brain activity of the particular individual;

g) utilizing a second processor configured, when executing a second set of software instructions stored in a second non-transient computer-readable hardware storage medium, to further perform at least the following additional operations:
1) projecting, in real time, the collected particular electrical signal data representative of the brain activity of the particular individual onto the pre-determined ordering of the denoised optimal set of wavelet packet atoms to obtain a particular set of projections of the particular individual;
2) normalizing, in real time, the particular set of projections of the particular individual using the pre-determined set of normalization factors to form a particular set of normalized projections of the particular individual;
3) applying at least one machine learning algorithm to the particular set of normalized projections of the particular individual to determine, in real time, at least one particular normalized projection in the particular set of normalized projections which corresponds to the at least one abnormal electrical brain activity, wherein the processor is configured to determine the at least one abnormal electrical brain activity from the particular set of normalized projections of the particular individual; and
4) generating, in real time, an indication of the at least one abnormal electrical brain activity of the particular individual.

In a particular embodiment of the above method, the method is used to detect the brain state of an individual. In a more particular embodiment, detecting the brain state of an individual may be used to optimize stimulation via optimized mixed reality or augmented reality.

In another particular embodiment, a method for detection of at least one of an environmentally-induced and an augmented reality-induced electrical brain activity in a mammal is presented, comprising:

a) utilizing a first electroencephalographic (EEG) monitoring device having a first set of three electrodes and applying the first set of three electrodes to particular points on a head of at least one individual, wherein the three electrodes of the first set are:
1) a first recording electrode,
2) a second recording electrode, and
3) a reference electrode;

b) collecting, by the first EEG monitoring device, a plurality of recordings of electrical signal data representative of brain activity of the at least one individual to form recorded electrical data;

c) utilizing a first processor configured, when executing a first set of software instructions stored in a first non-transient computer-readable hardware storage medium, to perform at least the following operations:
1) obtaining a pre-determined ordering of a denoised optimal set of wavelet packet atoms, by:
   i) obtaining an optimal set of wavelet packet atoms from the recorded digital representation of an electrical signal data from the recordings from the at least one individual, by:
      1) selecting a mother wavelet;
      2) determining an optimal set of wavelet packet atoms, by:
         a) deconstructing the recorded electrical signal data into a plurality of wavelet packet atoms, using the selected mother wavelet;
         b) storing the plurality of wavelet packet atoms in at least one first computer data object;
         c) determining the optimal set of wavelet packet atoms using the pre-determined mother wavelet and storing the optimal set of wavelet packet atoms in at least one second computer data object, wherein the determining is via utilizing a Coifman-Wickerhauser Best Basis algorithm;
   ii) denoising the obtained optimal set of wavelet packet atoms from the recordings from the at least one individual;
   iii) reordering the denoised optimal set of wavelet packet atoms from the recorded electrical signal data from the at least one individual to obtain a pre-determined ordering of the denoised optimal set of wavelet packet atoms, by determining a minimum path based on:
      1) projecting the recorded electrical signal data onto the denoised optimal set of wavelet packet atoms to obtain a set of projections,
         wherein a projection is a result of a convolution of an electrical signal in each time window of the signal and a wavelet packet atom;
      2) determining a collection of wire lengths within the set of projections;
      3) storing the collection of wire lengths for the set of projections in at least one third computer data object;
      4) iteratively determining a plurality of (i) orders of the projections, and (ii) respective wire lengths, by:
         i) determining the wire length between every two projections by at least one of:
         1) determining either mean or sum of absolute distance of a statistical measure of an energy of each projection from adjacent projections, and
         2) 1—a correlation of every two projections onto the wavelet packet atoms; and
         ii) storing the wire length data in at least one fourth computer data object;
5) determining, from the plurality of respective wire lengths, a particular order of projections that minimizes either the mean or sum of the wire lengths across the projections, across each time window, and across the plurality of recordings;

d) defining a set of pre-determined normalization factors, and storing the pre-determined normalization factors in at least one fifth computer data object;

e) utilizing a second EEG monitoring device having a second set of three electrodes and applying the second set of three electrodes to the particular points on a head of a particular individual;

f) collecting, by the second EEG monitoring device, in real-time, a recording of particular electrical signal data representative of brain activity of the particular individual;

g) utilizing a second processor configured, when executing a second set of software instructions stored in a second non-transient computer-readable hardware storage medium, to further perform at least the following additional operations:
1) projecting, in real time, the collected particular electrical signal data representative of the brain activity of the particular individual onto the pre-determined ordering of the denoised optimal set of wavelet packet atoms to obtain a particular set of projections of the particular individual;
2) normalizing, in real time, the particular set of projections of the particular individual using the pre-determined set of normalization factors to form a particular set of normalized projections of the particular individual;
3) applying at least one machine learning algorithm to the particular set of normalized projections of the particular individual to determine, in real time, at least one particular normalized projection in the particular set of normalized projections which corresponds to the at least one of the environmentally-induced and the augmented reality-induced electrical brain activity, wherein the processor is configured to determine the at least one of the environmentally-induced and the augmented reality-induced electrical brain activity from the particular set of normalized projections of the particular individual; and
4) generating, in real time, an indication of the at least one of the environmentally-induced and the augmented reality-induced electrical brain activity of the particular individual, wherein the indication triggers a feedback loop in at least one parameter controlling augmented reality.

In another embodiment of the method, the at least one individual and the particular individual are the same individual.

In yet another embodiment of the method, the collecting, by the first EEG monitoring device, the plurality of recordings of electrical signal data representative of brain activity is performed a plurality of times on the at least one individual.

In a further embodiment of the method, the applying the first set of three electrodes to particular points on the head of the at least one individual and the collecting, by the first EEG monitoring device, the plurality of recordings of electrical signal data representative of brain activity is performed a plurality of times on the at least one individual.

In another embodiment of the method, the plurality of recordings of electrical signal data representative of brain activity is at least 100 recordings of electrical signal data representative of brain activity.

Reference is now made to the following examples, which together with the above descriptions illustrate some embodiments of the invention in a non-limiting fashion.

### Illustrative Examples in Accordance with at Least Some Embodiments of the Present Invention

### Example 1: Optimized Mixed Reality in a Real Life Scenario: The Airport

The following represents aspects of the proposed invention:

In an embodiment, a subject (e.g., a person) is in an airport environment wearing augmented reality goggles. The at least one device configured to measure brain activity in the person and collect particular electrical signal data representative of the brain activity of the person (a brain activity sensor which may, e.g., be incorporated into the augmented reality goggles) senses that the person is stressed. Upon detection of stress in the person, the processor (or content suggestion system) chooses to present arrows indicating the locations of airport facilities that may provide comfort to the person, thereby reducing stress levels in the person. Under such a circumstance, the content suggestion system may choose to present arrows indicating the locations of the nearest bathrooms, eateries, and/or location of the flight information booth. In a more particular embodiment, the content suggestion system may choose to present arrows indicating the locations of a restaurant or bar that serves food or drinks preferred by the person under stress. The content suggestion system may further present arrows directing the person under stress to locations assigned to the particular airline on which the person is flying or, for example, the person's departure gate.

In a further embodiment, the at least one device configured to measure brain activity in the person and collect particular electrical signal data representative of the brain activity of the person (or brain activity sensor) senses that the person is excited and actively engaged in the environment of the airport. Under circumstances wherein the brain activity sensor senses excitement, engagement, or heightened interest, the content suggestion system may choose to present arrows directing the person to, for example, shopping destinations within the airport. Such shopping destinations may include duty free shops and may be accompanied by advertisements about specific items on sale. Advertisements may further be selected based on product preferences that can be determined based on, for example, the user's shopping profile. In a particular embodiment wherein the user has a history of purchasing perfumes, for example, the processor or content suggestion system could direct the user to perfumes that are on sale, especially those for which the user has a buying preference.

If there appears to be little brain excitement responsive to the indication of the arrows pointing to, for example, the duty free shops, the content suggestion system may choose to provide alternative information, including without limitation, general flight information, news, or other information based on the user's profile preferences.

In another embodiment, if there appears to be an increase in brain excitement responsive to an advertisement of a new bottle of Scotch Whiskey, an arrow indicating the location of a duty free shop that sells alcohol will be presented. The change in suggestion provided by the processor or content suggestion system is effectuated without needing active engagement on the part of the user. In other words, the content suggestion system detects the user's engagement/excitement and automatically alters the suggestions it provides to suit the user's responses.

Accordingly, optimized mixed reality as described herein does not require the user to provide feedback responsive to the content suggestion system in an active, deliberate manner by way of, for example, clicking “like” or “dislike” indicators, but rather obviates the need for such potential distractions on the part of the user by sensing the user's brain activity responsive to suggestions made by the content suggestion system.

### Example 2: Optimized Mixed Reality Relating to Changing Perceived Acceleration/Gravitational Force

A subject sitting on a chair that rotates at a certain speed around an axis at or outside the center of the chair, wherein the angle of the chair with respect to the rotating axis affects the gravitational force and the perceived weight and acceleration of the subject. In this embodiment, the angle of the chair may be altered to alter the subject's perceived weight and acceleration. Such an embodiment is applicable to, for example, flight simulation exercises, such as those performed by gamers and pilots.

### Example 3: Temperature and Other Environmental Changes

In an embodiment, temperature and other environmental changes may be implemented. A subject may be sitting or standing, walking or running (treadmill with speed and slope changing) inside a self-driving capsule surrounded by audio/visual and other sensory stimulation, such as those affecting temperature and humidity, smell, drinking fluids whose taste can be affected. All these parameters can be changed based on the brain and other physiological readings from the subject. For example, the type and volume of the music can be changed based on speed and slope of the treadmill and its effect on the heart rate, perspiration, and other physiological parameters to reduce heart rate for a given physical challenge via altering the musical stimulation.

In a closed loop embodiment, the parameter is modified following the change in heart rate to lead to some optimal parameter given all other environmental and physiological parameters. Such ‘optimal’ parameters are recorded for future stimulation. In one embodiment it is found that, for example, a certain temperature is optimal for activity at a certain physical challenge, and/or a certain type of music as well as visual stimulation and/or brain stimulation presents an optimized augmented reality environment conducive to peak performance.

### Example 4: Altering Stress Levels and Promoting Concentration

In an embodiment, a change in background music may be used to affect stress (e.g., reduce stress) and concentration [as is read from brain activity features (BAFs)] during a challenging cognitive task as is measured by BAFs or by an external evaluator of the subject's task, such as an evaluator of the difficulty of the content that is being read, or the cognitive challenge of a game.

In a text embodiment, a person is searching the web for “inspiring poetry”. The depth and complexity of the poetry presented is correlated to the brain state of the subject searching any may be varied based on the stress levels and/or and cognitive state of the subject. For example, more complicated poetry is presented when the subject is determined to be in a more aroused cognitive activity state.

In another embodiment, news items chosen from the preferred items of the subject are reordered based on the cognitive and emotional state of the subject. For example, more scientific subject matter is presented during higher brain cognitive arousal states.

In another embodiment, instructions for a specific type of meditation are provided based on person's emotional and cognitive state. For example, compassion meditation is suggested during low mood (depression) states which can be determined via brain activity state, or hormone levels, such as, e.g., cortisol or oxytocin levels. Relaxing meditation can be suggested when the subject is exhibiting BAFs characteristic of high stress levels.

In another embodiment, tactile stimulation is provided as needed. In some cases of fronto-temporal dementia (FTD) or Parkinson's disease, brain activity may be higher than normal (also known in the art as increased default mode network). In such cases, it is important to provide feedback to the subject to reduce such activity since prolonged high activity may be associated with brain dysfunction and cell death, because not enough oxygen is delivered to the brain to support the elevated activity. In such cases, vibrotactile stimulation, may be used to reduce the elevated activity by itself, or may alert the subject to get into a brain state that reduces the activity (for example, via a breathing meditation, walking, or looking at animal pictures on the phone).

In another embodiment, the objective is to elicit sexual arousal and pleasure in a subject. In this embodiment, sexual arousal and pleasure may be measured/assessed by brain states and hormone concentration and vibrator-induced stimulation can be modified based on reading of these parameters. Alternatively, or in addition, sexual arousal and pleasure may be enhanced further by presenting visual imagery expected to be or pre-determined to be of sexual interest to the subject.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

1. An augmented reality system, comprising:
(i) at least one augmented reality device, wherein the at least one augmented reality device is configured to create a plurality of augmented reality environments;
(ii) at least one device configured to measure brain activity of a subject and collect particular electrical signal data representative of the brain activity of the subject;
(iii) a processor configured, when executing a set of software instructions stored in a non-transient computer-readable hardware storage medium, to interpret brain activity of the subject, wherein the processor configured to interpret brain activity performs at least the following operations:
1) projecting, in real time, the collected particular electrical signal data representative of the brain activity of the subject onto a pre-determined ordering of a denoised optimal set of wavelet packet atoms to obtain a particular set of projections of the subject;
2) normalizing, in real time, the particular set of projections of the subject using the pre-determined set of normalization factors to form a particular set of normalized projections of the subject;
3) applying at least one machine learning algorithm to the particular set of normalized projections of the subject to determine, in real time, at least one particular normalized projection in the particular set of normalized projections which corresponds to the subject's electrical brain activity, wherein the processor is configured to determine the subject's electrical brain activity from the particular set of normalized projections of the subject;
4) generating, in real time, an indication of the electrical brain activity of the subject, wherein the indication correlates with an interpretation of the electrical brain activity of the subject; and
5) causing, based on the interpretation of the brain activity, a change in at least one parameter of the at least one augmented reality device, whereby the at least one augmented reality device is configured to alter, based on the change, a first augmented reality environment to present a second augmented reality environment to the subject,
thereby changing, in real time, the augmented reality environment in response to the subject's brain activity.

2. The augmented reality system of claim 1, further comprising at least one sensor device configured to measure a physiological response distinct from brain activity.

3. The augmented reality system of claim 2, wherein the physiological response distinct from brain activity comprises at least one of heart rate, heart rate variability, temperature, oxygenation, galvanic skin response (GSR), electro dermal activity, perspiration, olfactory response, blood sugar levels, or any combination thereof.

4. The augmented reality system of claim 2, wherein causing the change in the at least one parameter of the at least one augmented reality device is further based on measurements of the physiological responses distinct from brain activity and indicative of the physiological state of the subject.

5. The augmented reality system of claim 1, wherein the processor configured to interpret brain activity detects an emotional state of the subject.

6. The augmented reality system of claim 5, wherein the emotional state of the subject comprises at least one of a mood, wherein the mood comprises at least one of acceptance, joy, interest, anger, disgust, fear, sorrow, surprise, anxiety, or depression.

7. The augmented reality system of claim 6, wherein the interest correlates with at least one of an elevated degree of engagement relative to an average degree of engagement for the subject or a reduced degree of engagement relative to the average degree of engagement for the subject.

8. The augmented reality system of claim 7, wherein the degree of engagement correlates with a response to at least one of a real world stimulus or an augmented reality stimulus.

9. The augmented reality system of claim 1, further comprising a stimulating device, wherein the stimulating device is configured to stimulate the subject's brain to alter the subject's brain activity or alter a feature presented in an augmented reality environment.

10. The augmented reality system of claim 9, wherein the feature presented by the augmented reality environment comprises a text, a photograph, a video, or a combination thereof.

11. The augmented reality system of claim 4, further comprising a stimulating device, wherein the stimulating device is configured to stimulate the subject's brain to alter the subject's brain activity or alter a feature presented in an augmented reality environment.

12. The augmented reality system of claim 11, wherein the feature presented by the augmented reality environment comprises a text, a photograph, a video, or a combination thereof.

13. The augmented reality system of claim 1, wherein the at least one augmented reality device comprises at least one of a visual augmented reality device, an auditory augmented reality device, a tactile augmented reality device, or a gravitational augmented reality device.

14. The augmented reality system of claim 13, wherein the at least one of the visual augmented reality device comprises glasses, goggles, or contact lenses; the at least one of the auditory augmented reality device comprises ear buds or headphones; the at least one of the tactile augmented reality device comprises a vibrator or stimulator; and the at least one of the gravitational augmented reality device comprises a chair or simulation capsule.

15. The augmented reality system of claim 1, further comprising an environmental sensor device, wherein the environmental sensor device is configured to detect environmental features comprising temperature, sound volume, or light intensity.

16. The augmented reality system of claim 1, further comprising a stimulating device, wherein the stimulating device is configured to stimulate a body part of the subject other than the brain.

17. The augmented reality system of claim 1, further comprising determining at least one effect of the change in the at least one parameter of the at least one augmented reality device by
(iv) measuring brain activity of the subject after the change in the at least one parameter of the at least one augmented reality device and collecting particular electrical signal data representative of the brain activity of the subject; and
(vi) interpreting the brain activity of the subject after the change in the at least one parameter of the at least one augmented reality device, wherein the interpreting is performed by the processor configured, when executing the set of software instructions stored in the non-transient computer-readable hardware storage medium, to interpret brain activity of the subject after the change in the at least one parameter of the at least one augmented reality device, wherein the processor configured to interpret brain activity performs at least the following operations:
1) projecting, in real time, the collected particular electrical signal data representative of the brain activity of the subject after the change in the at least one parameter of the at least one augmented reality device onto the pre-determined ordering of a denoised optimal set of wavelet packet atoms to obtain the particular set of projections of the subject after the change in the at least one parameter of the at least one augmented reality device;
2) normalizing, in real time, the particular set of projections of the subject after the change in the at least one parameter of the at least one augmented reality device using the pre-determined set of normalization factors to form a particular set of normalized projections of the subject after the change in the at least one parameter of the at least one augmented reality device;
3) applying at least one machine learning algorithm to the particular set of normalized projections of the subject after the change in the at least one parameter of the at least one augmented reality device to determine, in real time, at least one particular normalized projection in the particular set of normalized projections which corresponds to the subject's electrical brain activity after the change in the at least one parameter of the at least one augmented reality device, wherein the processor is configured to determine the subject's electrical brain activity after the change in the at least one parameter of the at least one augmented reality device from the particular set of normalized projections of the subject; and
4) generating, in real time, an indication of the electrical brain activity of the subject after the change in the at least one parameter of the at least one augmented reality device, wherein the indication correlates with an interpretation of the electrical brain activity of the subject after the change in the at least one parameter of the at least one augmented reality device; and
5) determining, based on the interpretation of the electrical brain activity of the subject after the change in the at least one parameter of the at least one augmented reality device, whether a second change in the at least one parameter of the at least one augmented reality device would improve the augmented reality environment for the subject.

18. The augmented reality system of claim 17, wherein content of the augmented reality environment changes based on the interpretation of the electrical brain activity of the subject after the change in the at least one parameter of the at least one augmented reality device.

19. A method for improving augmented reality, comprising:
(i) receiving, by a processor, particular electrical signal data representative of brain activity of a subject exposed to at least one first augmented reality environment, wherein the at least one first augmented reality environment has been generated by at least one augmented reality device;
(iii) projecting, by the processor, in real time, the collected particular electrical signal data representative of the brain activity of the subject onto a pre-determined ordering of a denoised optimal set of wavelet packet atoms to obtain a particular set of projections of the subject;
(iv) normalizing, by the processor, in real time, the particular set of projections of the subject using the pre-determined set of normalization factors to form a particular set of normalized projections of the subject;
(v) applying, by the processor, at least one machine learning algorithm to the particular set of normalized projections of the subject to determine, in real time, at least one particular normalized projection in the particular set of normalized projections which corresponds to the subject's electrical brain activity, wherein the processor determines the subject's electrical brain activity from the particular set of normalized projections of the subject;
(vi) generating, by the processor, in real time, an indication of the electrical brain activity of the subject, wherein the indication correlates with an interpretation of the electrical brain activity of the subject; and
(vii) causing, by the processor, based on the interpretation of the brain activity, a change in at least one parameter of the at least one augmented reality device, whereby the at least one first augmented reality environment is changed to present at least one second augmented reality environment to the subject,
thereby improving the augmented reality environment in response to the subject's brain activity.

20. The method of claim 19, further comprising determining at least one effect of the change in the at least one parameter of the at least one augmented reality device by
(iv) measuring brain activity of the subject after the change in the at least one parameter of the at least one augmented reality device and collecting particular electrical signal data representative of the brain activity of the subject; and
(vi) interpreting the brain activity of the subject after the change in the at least one parameter of the at least one augmented reality device, wherein the interpreting, by the processor, when executing the set of software instructions stored in the non-transient computer-readable hardware storage medium, interprets brain activity of the subject after the change in the at least one parameter of the at least one augmented reality device, wherein the processor interprets brain activity by performing at least the following operations:
1) projecting, by the processor, in real time, the collected particular electrical signal data representative of the brain activity of the subject after the change in the at least one parameter of the at least one augmented reality device onto the pre-determined ordering of a denoised optimal set of wavelet packet atoms to obtain the particular set of projections of the subject after the change in the at least one parameter of the at least one augmented reality device;
2) normalizing, by the processor, in real time, the particular set of projections of the subject after the change in the at least one parameter of the at least one augmented reality device using the pre-determined set of normalization factors to form a particular set of normalized projections of the subject after the change in the at least one parameter of the at least one augmented reality device;
3) applying, by the processor, at least one machine learning algorithm to the particular set of normalized projections of the subject after the change in the at least one parameter of the at least one augmented reality device to determine, in real time, at least one particular normalized projection in the particular set of normalized projections which corresponds to the subject's electrical brain activity after the change in the at least one parameter of the at least one augmented reality device, wherein the processor determines the subject's electrical brain activity after the change in the at least one parameter of the at least one augmented reality device from the particular set of normalized projections of the subject; and
4) generating, by the processor, in real time, an indication of the electrical brain activity of the subject after the change in the at least one parameter of the at least one augmented reality device, wherein the indication correlates with an interpretation of the electrical brain activity of the subject after the change in the at least one parameter of the at least one augmented reality device; and
5) determining, by the processor, based on the interpretation of the electrical brain activity of the subject after the change in the at least one parameter of the at least one augmented reality device, whether a second change in the at least one parameter of the at least one augmented reality device caused a desired change in the at least one of the brain activity or other physiological parameters, thereby improving the augmented reality environment for the subject.
